(19) United States
(12) Patent Application Publication
(10) Pub. No.: US 2012/0245510 A1
(43) Pub. Date: Sep. 27, 2012

(54) ADHESIOLYSIS SYSTEM

(76) Inventor: Stephen R. Rakower, Corona del Mar, CA (US)

(21) Appl. No.: 13/308,471

(22) Filed: Nov. 30, 2011

Related U.S. Application Data
(60) Provisional application No. 61/466,373, filed on Mar. 22, 2011.

(51) Int. Cl. A61M 13/00

(52) U.S. Cl. 604/26

(57) ABSTRACT

Methods and apparatus for creating and maintaining a progressive pneumoperitoneum over a prolonged duration of time are provided. In some aspects, an apparatus may include a first elongate member having a proximal end, a distal end, and a first lumen extending from the distal end to the proximal end. The apparatus may further include a fixation member disposed between the distal end and the proximal end and configured to secure the apparatus relative to the abdominal wall of a patient. The apparatus may further include a pressure sensor in fluid communication with the first lumen configured to facilitate obtaining an intraperitoneal pressure measurement.

The present application claims the benefit of U.S. Provisional Patent Application Ser. No. 61/466,373, entitled “ADHESIOLYSIS SYSTEM,” filed on Mar. 22, 2011, which is hereby incorporated by reference in its entirety for all purposes.

### FIELD

The present invention generally relates to methods and apparatus for treating fibrotic adhesions in the body.

### BACKGROUND

Any abdominal surgery, especially open abdominal surgery is the most frequent cause of abdominal adhesions, with almost every patient undergoing open abdominal surgery developing adhesions. Abdominal adhesions are bands of scar tissue that form between abdominal tissues and organs that cause tissues and organs to adhere together. Adhesions can become tighter as time passes, causing problems years after surgery. Surgery-induced causes of abdominal adhesions include: tissue incisions, the handling of internal organs, the drying out of internal organs and tissues, contact of internal tissues with foreign materials, and fibrin from blood clots.

Conventional methods for treatment of symptomatic abdominal adhesions include surgery. Subsequent surgery in the abdomen, however, increases the risk that additional adhesions will occur, thus leading to more surgery. In addition each subsequent additional surgery is typically more difficult and dangerous than the previous surgery.

### SUMMARY

Various aspects of the subject technology, provide a method and apparatus for lysing and/or stretching fibrotic adhesions in the body by creating and maintaining a progressive pneumoperitoneum over a prolonged duration of time.

In various embodiments, methods of the technology include the following: Item 1. A method for creating and maintaining a pneumoperitoneum, the method comprising: selecting a patient with prior abdominal surgery and who is suffering form severe abdominal adhesions; inserting a device into the peritoneal cavity of the patient, the device comprising: (a) a first elongate member having a proximal end, a distal end, and a first lumen extending from the distal end to the proximal end; (b) a fixation member disposed between the distal end and the proximal end and configured to secure the device relative to the abdominal wall of the patient; and (c) a pressure sensor in fluid communication with the first lumen configured to facilitate obtaining an intraperitoneal pressure measurement; securing the device to the abdominal wall via the fixation member; creating a pneumoperitoneum by insufflating the peritoneal cavity with a gas through the first lumen; measuring an intraperitoneal pressure via the pressure sensor; displaying the measured intraperitoneal pressure; and maintaining or increasing the pneumoperitoneum for at least eight hours by insufflating the peritoneal cavity with additional gas through the first lumen.

Item 2. The method of Item 1, further comprising at least one of maintaining and increasing the pneumoperitoneum for at least 12 hours by insufflating the peritoneal cavity with additional gas through the first lumen.

Item 3. The method of Item 1, further comprising at least one of maintaining and increasing the pneumoperitoneum for at least 24 hours by insufflating the peritoneal cavity with additional gas through the first lumen.

Item 4. The method of Item 1, further comprising at least one of maintaining and increasing the pneumoperitoneum for at least 7 days by insufflating the peritoneal cavity with additional gas through the first lumen.

Item 5. The method of Item 1, further comprising at least one of maintaining and increasing the pneumoperitoneum for at least 10 days by insufflating the peritoneal cavity with additional gas through the first lumen.

Item 6. The method of Item 1, wherein the fixation member comprises a balloon.

Item 7. The method of Item 6, wherein the securing the device comprises inflating the balloon with a fluid.

Item 8. The method of Item 1, wherein the first elongate member has a length extending from the distal end to the proximal end, and the fixation member is spaced apart from the distal end by at least ⅙ of the length.

Item 9. The method of Item 1, wherein the first elongate member has a length extending from the distal end to the proximal end, and the fixation member is spaced apart from the distal end by at least ¼ of the length.

Item 10. The method of Item 1, wherein the fixation member is spaced apart from the distal end by at least 10 centimeters.

Item 11. The method of Item 1, further comprising sealing the area between the outer surface of the first elongate member and the opening into the peritoneal cavity of the patient with a cover.

Item 12. The method of Item 11, wherein the cover comprises a balloon.

Item 13. The method of Item 1, further comprising a valve, located at the proximal end, that adjusts a flow of gas through the first elongate member.

Item 14. The method of Item 1, wherein the first elongate member comprises a taper that narrows from proximally to distally, thereby facilitating insertion of the first elongate member into the abdomen of the patient.

Item 15. The method of Item 1, wherein the first elongate member has a diameter of about 5 millimeters or less.

Item 16. The method of Item 1, wherein the first elongate member has a diameter of about 2 millimeters or less.

Item 17. The method of Item 1, wherein a distal portion of the first elongate member comprises a plurality of openings for dissemination of the gas into the peritoneal cavity.

Item 18. The method of Item 1, wherein the device further comprises a second elongate member having a proximal portion, a distal portion, and a second lumen extending within the second elongate member.

Item 19. The method of Item 18, wherein the first and second lumens are in fluid communication with each other.

Item 20. The method of Item 18, wherein the first and second lumens are in fluid communication with each other proximal to the fixation member.

Item 21. The method of Item 18, wherein the pressure sensor is in fluid communication with the second lumen.

Item 22. The method of Item 1, further comprising by a flow meter in fluid communication with the first lumen, measuring a volume of gas flowing through the first lumen.

Item 23. The method of Item 1, further comprising by a counter in fluid communication with the first lumen, counting the number of times the gas flows through the first lumen.

Item 24. The method of Item 1, further comprising placing the device into the peritoneal cavity under image guidance by at least one of ultrasound, CT, MRI, and fluoroscopy.

Item 25. An apparatus for creating and maintaining a pneumoperitoneum, the apparatus comprising: a first elongate member having a proximal end, a distal end, and a first lumen extending from the distal end to the proximal end; a fixation member disposed between the distal end and the proximal end and configured to secure the apparatus relative to the abdominal wall of a patient; a pressure sensor in fluid communication with the first lumen configured to facilitate obtaining an intraperitoneal pressure measurement; a valve, located at the proximal end and configured to adjust a flow of gas through the first elongate member; and a flow meter in fluid communication with the first lumen and configured to measure a volume of gas flowing through the first lumen.

Item 26. The apparatus of Item 25, wherein the fixation member comprises a balloon.

Item 27. The apparatus of Item 26, wherein the balloon is inflatable with a fluid.

Item 28. The apparatus of Item 25, wherein the first elongate member has a length extending from the distal end to the proximal end, and the fixation member is spaced apart from the distal end by at least ⅙ of the length.

Item 29. The apparatus of Item 25, wherein the first elongate member has a length extending from the distal end to the proximal end, and the fixation member is spaced apart from the distal end by at least ¼ of the length.

Item 30. The apparatus of Item 25, wherein the fixation member is spaced apart from the distal end by at least 10 centimeters.

Item 31. The apparatus of Item 25, further comprising a cover configured to seal the area between the outer surface of the first elongate member and the opening into the peritoneal cavity of the patient.

Item 32. The apparatus of Item 31, wherein the cover comprises a balloon.

Item 33. The apparatus of Item 25, wherein the first elongate member comprises a taper that narrows from proximally to distally, thereby facilitating insertion of the first elongate member into the abdomen of the patient.

Item 34. The apparatus of Item 25, wherein the first elongate member has a diameter of about 5 millimeters or less.

Item 35. The apparatus of Item 25, wherein the first elongate member has a diameter of about 2 millimeters or less.

Item 36. The apparatus of Item 25, wherein a distal portion of the first elongate member comprises a plurality of openings for dissemination of the gas into the peritoneal cavity.

Item 37. The apparatus of Item 25, further comprising a second elongate member having a proximal portion, a distal portion, and a second lumen extending within the second elongate member.

Item 38. The apparatus of Item 37, wherein the first and second lumens are in fluid communication with each other.

Item 39. The apparatus of Item 37, wherein the first and second lumens are in fluid communication with each other proximal to the fixation member

Item 40. The apparatus of Item 37, wherein the pressure sensor is in fluid communication with the second lumen.

Item 41. The apparatus of Item 25, wherein the first elongate member is at least partially radiopaque.

Item 42. An apparatus for creating and maintaining a pneumoperitoneum, the apparatus comprising: a first elongate member having a proximal end, a distal end, and a first lumen extending from the distal end to the proximal end; means for securing the apparatus relative to the abdominal wall of a patient; a valve located at the proximal end and configured to adjust a flow of gas through the first elongate member; means for measuring an intraperitoneal pressure; and means for measuring a volume of gas flowing through the first lumen.

Item 43. The apparatus of Item 42, wherein the means for securing comprises a balloon disposed between the distal end and the proximal end.

Item 44. The apparatus of Item 43, wherein the first elongate member has a length extending from the distal end to the proximal end, and the balloon is spaced apart from the distal end by at least ⅙ of the length.

Item 45. The apparatus of Item 42, wherein the first elongate member has a length extending from the distal end to the proximal end, and the balloon is spaced apart from the distal end by at least ¼ of the length.

Item 46. The apparatus of Item 42, wherein the balloon is spaced apart from the distal end by at least 10 centimeters.

Item 47. The apparatus of Item 42, wherein the balloon is inflatable with a fluid.

Item 48. The apparatus of Item 42, further comprising a cover configured to seal the area between the outer surface of the first elongate member and the opening into the peritoneal cavity of the patient.

Item 49. The apparatus of Item 48, wherein the cover comprises a balloon.

Item 50. The apparatus of Item 42, wherein the first elongate member comprises a taper that narrows from proximally to distally, thereby facilitating insertion of the first elongate member into the abdomen of the patient.

Item 51. The apparatus of Item 42, wherein the first elongate member has a diameter of about 5 millimeters or less.

Item 52. The apparatus of Item 42, wherein the first elongate member has a diameter of about 2 millimeters or less.

Item 53. The apparatus of Item 42, wherein a distal portion of the first elongate member comprises a plurality of openings for dissemination of the gas into the peritoneal cavity.

Item 54. The apparatus of Item 42, wherein the means for measuring an intraperitoneal pressure comprises a pressure sensor in fluid communication with the first lumen.

Item 55. The apparatus of Item 42, wherein the means for measuring an intraperitoneal pressure comprises a second elongate member having a proximal portion, a distal portion, and a second lumen extending within the second elongate member, the distal portion configured to facilitate obtaining an intraperitoneal pressure measurement displayed at the proximal portion.

Item 56. The apparatus of Item 55, wherein the first lumen and second lumen are in fluid communication with each other.

Item 57. The apparatus of Item 55, wherein the first lumen and second lumen are in fluid communication with each other proximal to the fixation member.

Item 58. The apparatus of Item 42, wherein the means for measuring a volume of gas flowing through the first lumen comprises a flow meter in fluid communication with the first lumen and configured to measure a volume of gas flowing through the first lumen.

Item 59. The apparatus of claim 42, wherein the means for measuring a volume of gas flowing through the first lumen comprises a counter in fluid communication with the first lumen and configured to count the number of times the gas flows through the first lumen.

Item 60. The apparatus of Item 42, wherein the first elongate member is at least partially radiopaque.

Item 61. A method for treating fibrotic adhesions in a patient, the method comprising: selecting a patient who has developed significant fibrotic adhesions between segments of the patient's intestine following a first abdominal surgery; creating a pneumoperitoneum by insufflating the patient's peritoneal cavity with a gas; maintaining and/or increasing the pneumoperitoneum for at least eight hours by insufflating the peritoneal cavity with additional gas; and performing a second abdominal surgery on the patient.

Item 62. The method of Item. 61, further comprising at least one of maintaining and increasing the pneumoperitoneum for at least 12 hours by insufflating the peritoneal cavity with additional gas.

Item 63. The method of Item 61, further comprising at least one of maintaining and increasing the pneumoperitoneum for at least 24 hours by insufflating the peritoneal cavity with additional gas.

Item 64. The method of Item 61, further comprising at least one of maintaining and increasing the pneumoperitoneum for at least 7 days by insufflating the peritoneal cavity with additional gas.

Item 65. The method of Item 61, further comprising at least one of maintaining and increasing the pneumoperitoneum for at least 10 days by insufflating the peritoneal cavity with additional gas

Additional features and advantages of the subject technology will be set forth in the description below, and in part will be apparent from the description, or may be learned by practice of the subject technology. The advantages of the subject technology will be realized and attained by the structure particularly pointed out in the written description and claims hereof as well as the appended drawings.

It is to be understood that both the foregoing general description and the following detailed description are exemplary and explanatory and are intended to provide further explanation of the subject technology as claimed.

### BRIEF DESCRIPTION OF THE DRAWINGS

The accompanying drawings, which are included to provide further understanding of the subject technology and are incorporated in and constitute a part of this specification, illustrate aspects of the subject technology and together with the description serve to explain the principles of the subject technology.

FIG. 1 illustrates a schematic of an apparatus for creating and maintaining a pneumoperitoneum, in accordance with various aspects of the subject technology.

FIG. 2 illustrates an example of an apparatus for creating and maintaining a pneumoperitoneum, in accordance with various aspects of the subject technology.

FIG. 3 illustrates an example of an apparatus for creating and maintaining a pneumoperitoneum with one or more valves, in accordance with various aspects of the subject technology.

FIG. 4 illustrates an example of an apparatus for creating and maintaining a pneumoperitoneum, in accordance with various aspects of the subject technology.

FIG. 5 illustrates an example of an apparatus for creating and maintaining a pneumoperitoneum with concentric elongate members, in accordance with various aspects of the subject technology.

FIG. 6 illustrates an example of a method for creating and maintaining a pneumoperitoneum, in accordance with various aspects of the subject technology.

FIG. 7 illustrates an example of a method for treating fibrotic adhesions in a patient, in accordance with various aspects of the subject technology.

### DETAILED DESCRIPTION

In the following detailed description, numerous specific details are set forth to provide a full understanding of the subject technology. It will be apparent, however, to one ordinarily skilled in the art that the subject technology may be practiced without some of these specific details. In other instances, well-known structures and techniques have not been shown in detail so as not to obscure the subject technology. Like components are labeled with identical element numbers for ease of understanding.

Any abdominal surgery, especially open abdominal surgery is the most frequent cause of abdominal adhesions, with almost every patient undergoing open abdominal surgery developing adhesions. It is also understood that abdominal surgery may also include pelvic surgery. Adhesions can become tighter as time passes, causing problems years after surgery. Surgery-induced causes of abdominal adhesions include: tissue incisions, the handling of internal organs, the drying out of internal organs and tissues, contact of internal tissues with foreign materials, and fibrin from blood clots.

Conventional treatment of symptomatic abdominal adhesions involves surgery. Subsequent surgery in the abdomen, however, increases the risk that additional adhesions will occur, thus leading to more surgery.

Various aspects of the subject technology, provide a method and apparatus for lysing and/or stretching fibrotic adhesions in the body by creating and maintaining a progressive pneumoperitoneum over a prolonged duration of time. According to certain aspects of the subject technology, a surgeon caring for a patient with a severely adhesed “hostile” surgical abdomen, facing a “must operate” scenario, may intentionally create and maintain a progressive pneumoperitoneum over a prolonged period of time to render the subsequent necessary abdominal surgery easier, safer, and more effective. In some aspects, the intentional progressive pneumoperitoneum also reduces subsequent formation of adhesions. In some aspects, the patient has had prior abdominal surgery. In some aspects the subject technology may be used some time after surgery to prevent or impede progression of adhesions. Tools used for creating a pneumoperitoneum include the use of a transcutaneous instillation of air by blind needle placement, the use of a Veres needle, or a catheter. All of these tools have their potential disadvantages and are not designed specifically for creating and maintaining a progressive pneumoperitoneum over a prolonged duration of time.

According to various aspects of the subject technology, a method and apparatus for creating and maintaining a progressive pneumoperitoneum over a prolonged duration of time is provided, for example, by inserting a device into the peritoneal cavity of a patient, securing the device to the abdominal wall, creating a pneumoperitoneum, measuring the intraperitoneal pressure, displaying the intraperitoneal pressure, and maintaining or increasing the pneumoperitoneum by insufflating the peritoneal cavity with additional gas. In some aspects, the device may comprise a first elongate member having a proximal end, a distal end, and a first lumen extending from the distal end to the proximal end; a fixation member disposed between the distal end and the proximal end and configured to secure the device relative to the abdominal wall of the patient; and a pressure sensor in fluid communication with the first lumen configured to facilitate obtaining an intraperitoneal pressure measurement.

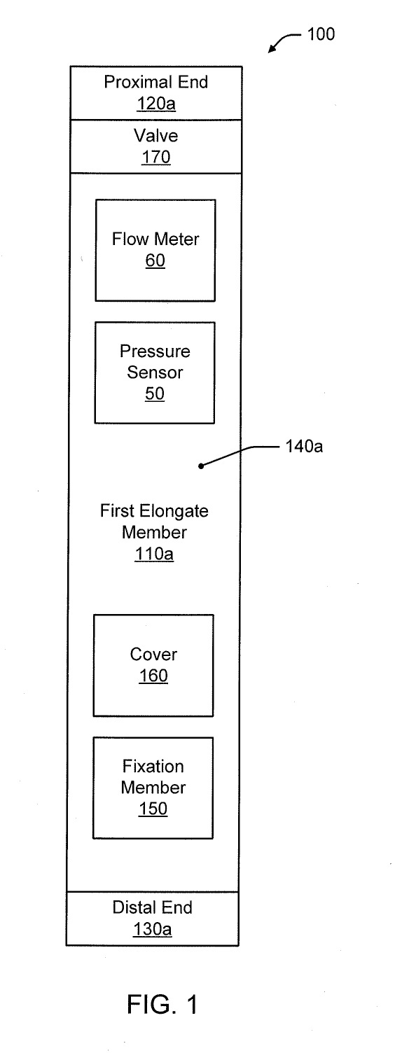

FIG. 1 illustrates a schematic of an apparatus 100 for creating and maintaining a pneumoperitoneum, in accordance with various aspects of the subject technology. The apparatus 100 may comprise a first elongate member 110a, a valve 170, a flow meter 60, a pressure sensor 50, a cover 160, and a fixation member 150. The first elongate member 110a may be configured for insertion into the abdominal cavity of a patient. The valve 170 may be configured to adjust a flow of gas. The flow meter may be configured to measure a volume of gas. The pressure sensor 50 may be configured to obtain an intraperitoneal pressure measurement. The cover 160 may be configured to secure the first elongate member 110a relative to the abdomen. The fixation member 150 may be configured to secure the apparatus 100 relative to the abdominal wall of the patient. In some aspects, the first elongate member 110a has a first lumen 140a to be used to insufflate the peritoneal cavity with the gas, thereby creating a pneumoperitoneum. In other aspects, the first lumen 140a is used to maintain or increase the pneumoperitoneum. According to some aspects, the pressure sensor 50 is in fluid communication with the first lumen 140a. The pressure sensor 50 may, for example, comprise a strain gauge, configured to facilitate obtaining an intraperitoneal pressure measurement.

In one aspect, the first elongate member 110a may comprise a proximal end 120a located away from the patient, a distal end 130a located towards the patient, and a first lumen 140a extending from the distal end 130a to the proximal end 120a. The first lumen 140a is adapted for the flow of gas, such as air, and is adapted to not chemically react or cause a reaction with the gas. The first elongate member 110a may be composed of one or more materials or composition, that is known to be safe for use on a patient. Such materials include, for example, silicon-based, polychloride-based, polymethacrylate-based, or other similar materials generally known by those having ordinary skill in the art. In some aspects, the first elongate member 110a is flexible.

In some aspects, the first elongate member 110a has a diameter of about 8 millimeters or less, or about 7 millimeters or less, or about 6 millimeters or less, or about 4 millimeters or less, or about 2 millimeters or less. In some aspects, the first elongate member 110a may be configured for insertion into the abdomen of the patient. For example, the distal end 130a of the first elongate member 110a may comprise a taper that narrows from proximally to distally, thereby facilitating insertion of the first elongate member 110a into the abdomen of the patient. Once the distal end 130a of the first elongate member 110a is inserted into the abdomen of the patient, a pneumoperitoneum may be created by insufflating the peritoneal cavity with a gas through the first lumen 140a. In some aspects, the first elongate member 110a is configured for effective dissemination of the gas into the peritoneal cavity. For example, the distal portion of the first elongate member 110a may comprise a plurality of openings for dissemination of the gas into the peritoneal cavity of the patient. The plurality of openings enables efficient distribution of the gas into the peritoneal cavity and also, reduces complications caused by any clogging of some of the openings which may occur during use. In some aspects, the first elongate member 110a is configured to maintain the gas inside the peritoneal cavity. For example, the proximal end 120a of the first elongate member 110a may be configured to receive a valve 170. The valve 170 is configured to prevent the gas of the peritoneal cavity from escaping through the first lumen 140a and out of the peritoneal cavity of the patient, as further described below. In other aspects, the first elongate member 110a may be configured to be placed partially inside the peritoneal cavity for a prolonged duration, for example, for at least eight hours. Although in this example, the first elongate member 110a is placed partially inside the peritoneal cavity for at least eight hours, the first elongate member 110a is capable of being placed partially inside the peritoneal cavity for a longer period of time, for example, for at least 12 hours, for at least 24 hours, for at least 7 days, for at least 10 days, for at least 20 days, and also for at least 30 days.

In some aspects, the first elongate member 110a may be configured to facilitate obtaining an intraperitoneal pressure measurement. For example, the distal end 130a of the first elongate member 110a may be located in an area inside the peritoneal cavity of the patient, enabling the first lumen 140a to be in fluid communication with the gas inside the peritoneal cavity, thereby allowing an intraperitoneal pressure measurement. In other aspects, a pressure sensor 50, such as a strain gauge, may be in fluid communication with the first lumen 140a, thereby allowing an intraperitoneal pressure measurement to be taken. In some aspects, the first elongate member 110a may be configured to display the intraperitoneal pressure measurement at a proximal portion of the first elongate member 110a. For example, the strain gauge may be connected to a pressure displaying device, thereby allowing a pressure measurement of the peritoneal cavity to be displayed. Measurement of the intraperitoneal pressure may, for example, enable a physician to monitor a pneumoperitoneum, determine whether additional gas should be insufflated into the peritoneal cavity, and whether the patient is receiving treatment well.

In some aspects, a fixation member 150 may be configured to secure the apparatus 100 relative to the abdominal wall of a patient. The fixation member 150 may be configured to be inserted through the abdominal wall and into the peritoneal cavity, for example, by maintaining an outer dimension that is substantially the same as the outer diameter of the first elongate member 110a. The fixation member 150, once inside the peritoneal cavity, may be configured to expand. For example, fixation member 150 may comprise a balloon configured to be inflatable with a fluid such as a gas or a liquid. Common examples of a liquid include water and saline, and for a gas, include air. Once inflated, the balloon expands to a dimension greater than the outer dimension of the first elongate member 110a, thereby securing the apparatus 100 to the abdominal wall of the patient. In some aspects, the shape of the balloon may be configured to maximize surface contact with the inside wall of the abdomen, such shape may, for example, be substantially spherical, conical, cylindrical, or elliptical. Although in this example the fixation member is a balloon, it is understood that other methods for securing the device relative to the abdominal wall of the patient are known and understood by a person of ordinary skill in the art.

In some aspects, the fixation member 150 may be disposed between the distal end 130a and the proximal end 120a of the first elongate member 110a. For example, wherein the first elongate member 110a has a length extending from the distal end 130a to the proximal end 120a, the fixation member 150 may be spaced apart from the distal end 130a by at least ⅙ of the length. In another example, wherein the first elongate member 110a has a length extending from the distal end 130a to the proximal end 120a, the fixation member 150 may be spaced apart from the distal end 130a by at least ¼ of the length. In yet another example the fixation member 150 may be spaced apart from the distal end 130a by at least 10 centimeters.

In some aspects, the valve 170 may be located at the proximal end 120a of the first elongate member 110a. The valve 170 may be configured to adjust the flow of gas through the first elongate member 110a. For example, the valve 170 may comprise a ball valve capable of achieving various flow rates for adjusting the amount of gas flowing through the valve 170. A pneumoperitoneum may be created by insufflating the peritoneal cavity with the gas through the first lumen 140a by adjusting the valve 170 to an open position to allow the gas to flow therethrough and enter into the peritoneal cavity. The pneumoperitoneum may be maintained by insufflating the peritoneal cavity with additional gas through the first lumen 140a by adjusting the valve 170 to an open position to allow the gas to flow therethrough and enter into the peritoneal cavity. In some aspects, the valve 170 may be adjusted to a closed position to keep the gas from leaking out of the peritoneal cavity. The pneumoperitoneum may be maintained, for example, for at least eight hours. Although in this example, the pneumoperitoneum is maintained for at least eight hours, the subject technology is capable of maintaining the pneumoperitoneum for a longer period of time. For example, the pneumoperitoneum may be maintained for at least 12 hours, for at least 24 hours, for at least 7 days, for at least 10 days, for at least 20 days, and also for at least 30 days. In some aspects, the valve 170 may be adjusted to allow the gas in the peritoneal cavity to flow out of the peritoneal cavity, thereby achieving an ideal pressure in the peritoneal cavity. In other aspects, the valve 170 may be opened to allow additional gas to flow into the peritoneal cavity, thereby increasing the pneumoperitoneum.

In some aspects, the pneumoperitoneum may be created, maintained, and increased by insufflating the peritoneal cavity with a gas through the first lumen 140a by using a syringe to inject the gas into the peritoneal cavity. The pneumoperitoneum may be maintained by insufflating the peritoneal cavity with additional gas through the first lumen 140a by using the syringe to inject gas into the peritoneal cavity. The pneumoperitoneum may be increased by insufflating the peritoneal cavity with additional gas through the first lumen 140a by using the syringe to inject additional gas into the peritoneal cavity.

In some aspects, a flow meter 60 may be configured to measure a volume of gas flowing through the first lumen 140a of the first elongate member 110a. For example, the flow meter may comprise a flow nozzle disposed at a proximal portion of the first elongate member 110a. The flow nozzle may be in fluid communication with the first lumen 140a, allowing measurement of the volume of gas flowing through the first lumen 140a. The measurement of the volume of gas flowing through the first lumen 140a and into the peritoneal cavity may, for example, enable a physician to determine whether gas is flowing into the peritoneal cavity.

In other aspects, an automatic counter may be disposed at a proximal portion of the first elongate member 110a to count the number of times a syringe injects gas into the first lumen. The total volume of gas insufflated into the peritoneal cavity may then be calculated by multiplying the value of the counter by the volume capacity of the syringe. For example, if a physician or nurse uses a 50 cc syringe to insufflate the peritoneal cavity, four injections of gas would be counted by the automatic counter. The total volume of gas insufflated into the peritoneal cavity would then be 200 cc in this example. Although the apparatus 100 may provide pressure and volume data to a physician for assisting the physician in assessing the pneumoperitoneum, monitoring and assessment of certain patient functions, such as ventilation, patient discomfort, and oxygenation, may also be beneficial.

In some aspects, the apparatus 100 may comprise a cover 160 configured to seal the insertion point in the abdominal wall, maintain the gas inside the peritoneal cavity, and secure the first elongate member 110a relative to the abdomen. After insertion of the first elongate member 110a into the abdominal wall and subsequent insufflating of the gas into the peritoneal cavity, the area between the outer surface of the first elongate member 110a and the opening into the peritoneal cavity of the patient may cause the gas to escape the peritoneal cavity. The cover 160 is configured to seal the area between the outer surface of the first elongate member and the opening into the peritoneal cavity of the patient. For example, the cover 160 may comprise a cap configured to receive the first elongate member 110a and prevent the gas from escaping the peritoneal cavity. In some aspects, the cap may be configured to be securely attached to the first elongate member 110a with, for example, a snap-fit or hardware. In some aspects, the cover 160 is configured to prevent the first elongate member 110a from sliding longitudinally toward and/or away from the abdomen, by for example, mechanically engaging a series of protrusions extending radially from the outer surface of the first elongate member 110a, the protrusions configured to secure the first elongate member 110a, the cover 160, and the abdomen relative to each other. In some aspects, the cover 160 may comprise a balloon, configured to be inflatable with a fluid such as a gas or liquid and configured to prevent the gas from escaping the peritoneal cavity.

In some aspects, the apparatus 100 may be configured for image guidance into the peritoneal cavity. For example, the first elongate member may be partially radiopaque to enable guidance of the distal end 130b and distal portion of the first elongate member 110a into the peritoneal cavity under image guidance by at least one of ultrasound, computed tomography (CT) scan, magnetic resonance imaging (MRI), and fluoroscopy. In other aspects, the apparatus 100 may be configured with an endoscopic system comprising, for example, a laparoscopic system for visually assessing the pneumoperitoneum from within the peritoneal cavity.

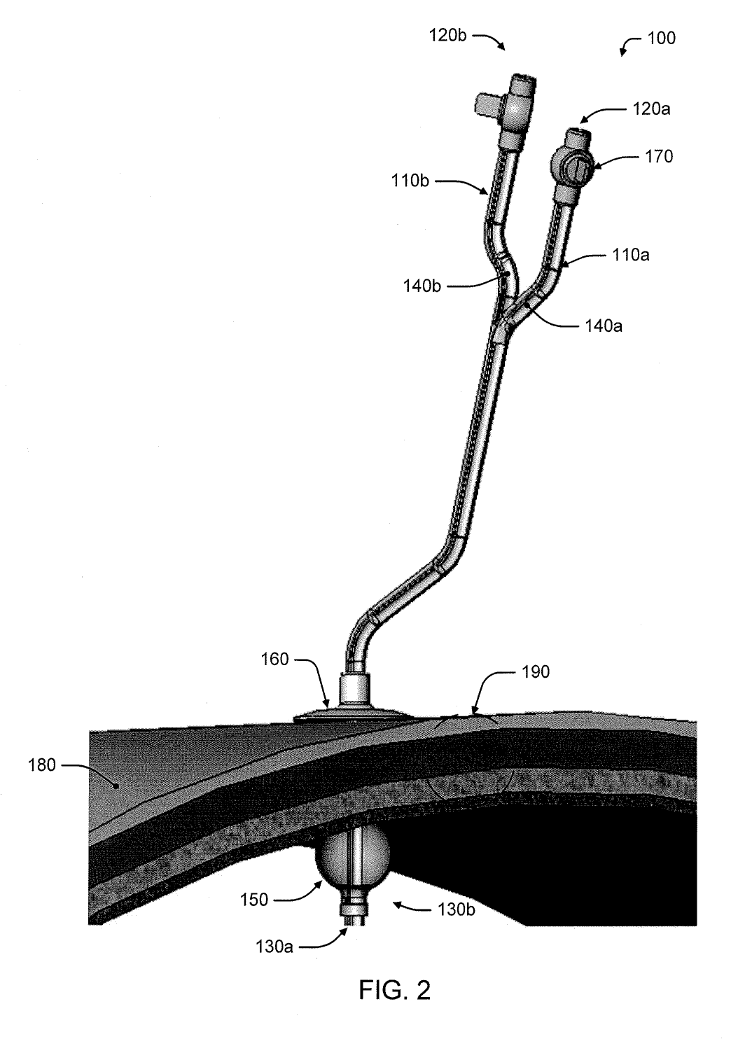

FIG. 2 illustrates an example of an apparatus 100 for creating and maintaining a pneumoperitoneum, in accordance with various aspects of the subject technology. The apparatus 100 may comprise a first elongate member 110a, a second elongate member 110b, a balloon 150, a valve 170, and a flow meter. The first elongate member 110a may be configured for insertion into the abdominal cavity of a patient 180. The second elongate member 110b may be configured to obtain an intraperitoneal pressure measurement. The balloon 150 may be configured to secure the apparatus 100 relative to the abdominal wall 190 of the patient 180. The valve 170 may be configured to adjust a flow of gas. The flow meter may be configured to measure a volume of gas.

In some aspects, the second elongate member 110b may comprise a proximal portion 120b located away from the patient, a distal portion 130b located towards the patient, and a second lumen 140b extending within the second elongate member 110b. The second lumen 140b may be adapted to contain gas, such as air, and is adapted to not chemically react or cause a reaction with the gas. The second elongate member 110b may be composed of one or more materials or composition, that is known to be safe for use on a patient. Such materials include, for example, silicon-based, polychloride-based, polymethacrylate-based, or other similar materials generally known by those having ordinary skill in the art.

In some aspects, the second elongate member 110b may be configured to facilitate obtaining an intraperitoneal pressure measurement. For example, the distal portion 130b of the second elongate member 110b may be located in an area inside the peritoneal cavity of the patient 180, enabling the second lumen 140b to be in fluid communication with the gas inside the peritoneal cavity, thereby allowing an intraperitoneal pressure measurement. In some aspects, the first lumen 140a of the first elongate member 110a and the second lumen 140b of the second elongate member 110b are in fluid communication with each other, thereby allowing an intraperitoneal pressure measurement. The first lumen 140a and the second lumen 140b may be in fluid communication with each other proximal to the balloon 150. In other aspects, a strain gauge may be connected to the second elongate member 110b, thereby allowing an intraperitoneal pressure measurement.

In some aspects, the second elongate member 110b may be configured to display the intraperitoneal pressure measurement at the proximal portion 120b of the second elongate member 110b. For example, a pressure gauge may be connected to a proximal end of the second elongate member 110b, thereby allowing a pressure measurement of the peritoneal cavity to be displayed. Measurement of the intraperitoneal pressure may, for example, enable a physician to monitor a pneumoperitoneum, determine whether additional gas should be insufflated into the peritoneal cavity, and whether the patient is receiving treatment well.

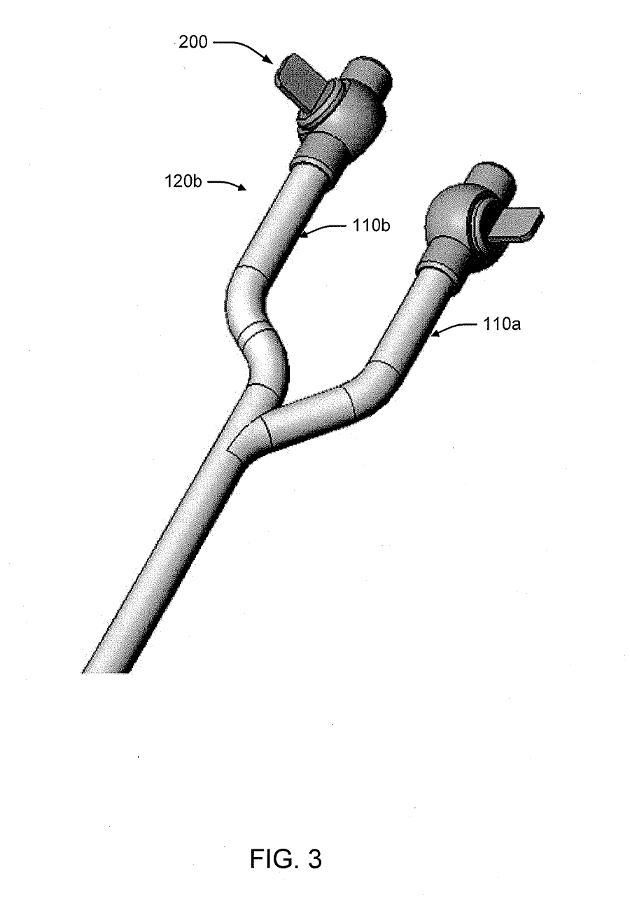

FIG. 3 illustrates an example of an apparatus for creating and maintaining a pneumoperitoneum with one or more valves, in accordance with various aspects of the subject technology. In some aspects, the second elongate member 110b may be configured to receive a valve 200 at the proximal portion 120b of the second elongate member 110b. In some aspects the valve 200 seals off the second elongate member 110b from a device, such as a pressure or volume sensor, including for example a strain gauge, manometer, or flowmeter.

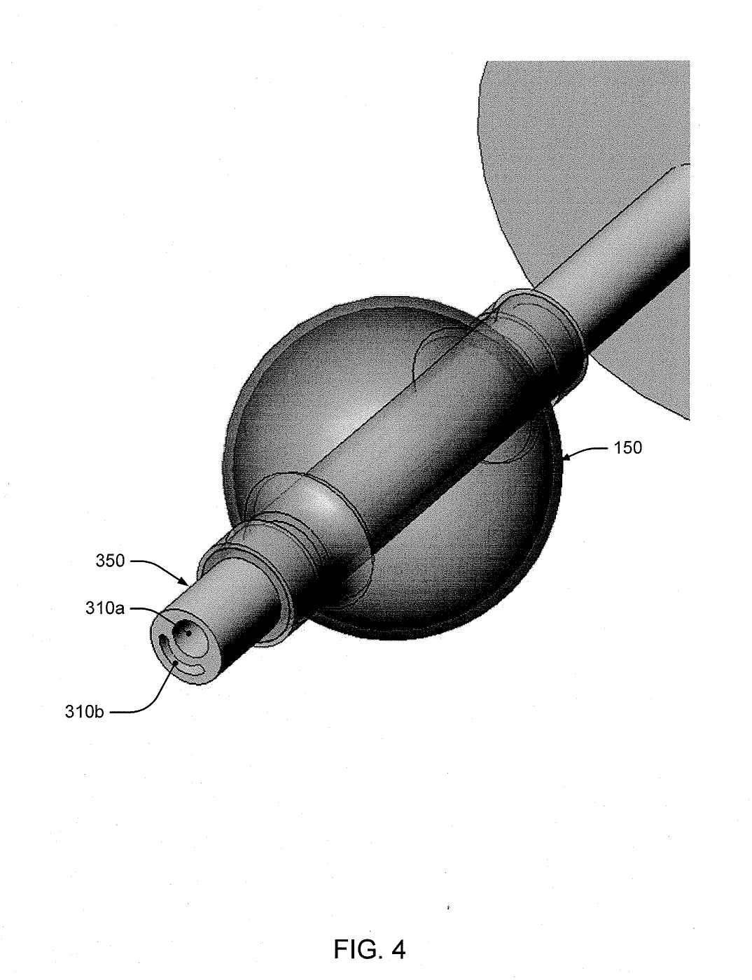

FIG. 4 illustrates an example of an apparatus for creating and maintaining a pneumoperitoneum, in accordance with various aspects of the subject technology. In some aspects, a first lumen 310a and a second lumen 310b may be disposed in elongate member 350. Elongate member 350 is configured to be inserted into the abdomen. The first lumen 310a may then be used to insufflate the peritoneal cavity with a gas through the first lumen 310a. In some aspects, the second lumen 310b is configured to facilitate obtaining an intraperitoneal pressure measurement. In other aspects, the first lumen 310a and the second lumen 310b may have differing cross sections, with the cross section of the first lumen 310a being larger than the cross section of the second lumen 310b, thereby reducing resistance of the gas as it flows through the first lumen 310a and into the peritoneal cavity.

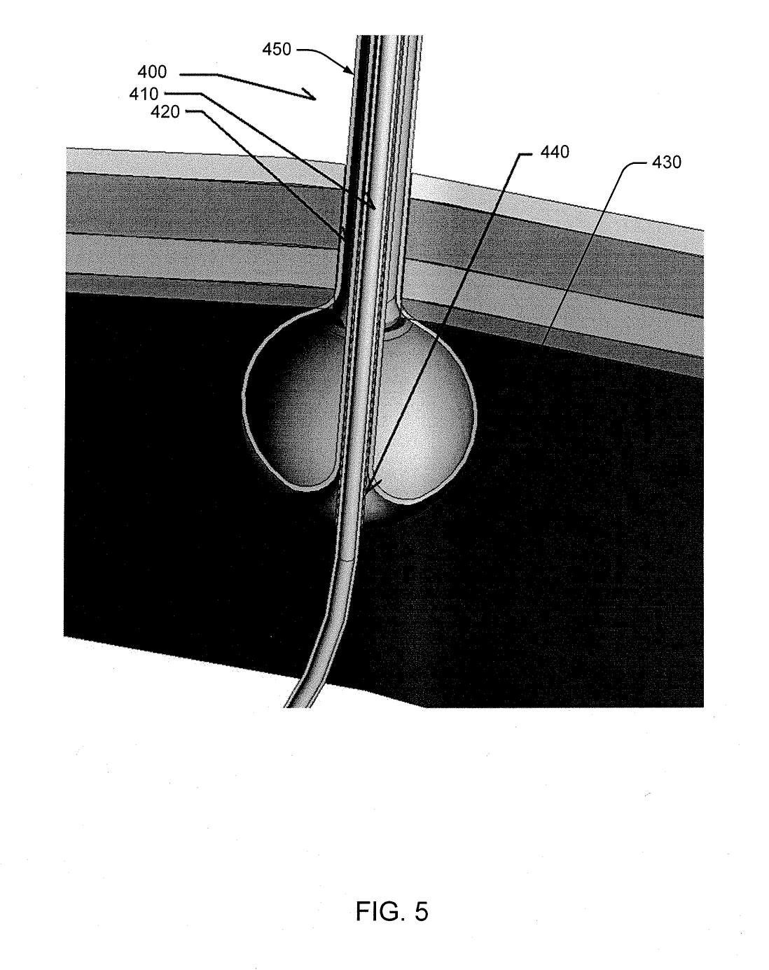

FIG. 5 illustrates an example of an apparatus 400 for creating and maintaining a pneumoperitoneum with concentric elongate members, in accordance with various aspects of the subject technology. The apparatus 400 may comprise a first elongate member 410, a second elongate member 440, and a balloon 420. The first elongate member 410 may be configured for insufflating the peritoneal cavity 430 with a gas. The second elongate member 440 may be configured for insertion into the abdominal cavity 430. The balloon 420 may be configured to secure the apparatus 400 relative to the abdominal wall of the patient. In some aspects, the first elongate member 410 is surrounded by a supporting member 440 such that supporting member 440 supports the first elongate member 410 as it is inserted into the abdomen of the patient. In some aspects, the balloon 420 may be inserted into the peritoneal cavity of the patient and inflatable with gas or liquid. The balloon 420 expands in the peritoneal cavity such that the apparatus 400 may not be removed from the peritoneal cavity. In some aspects, the balloon 420 is attached to a tubular portion 450 that is configured to expand in a radial direction, such that the tubular portion 450 seals the gas contained in the peritoneal cavity.

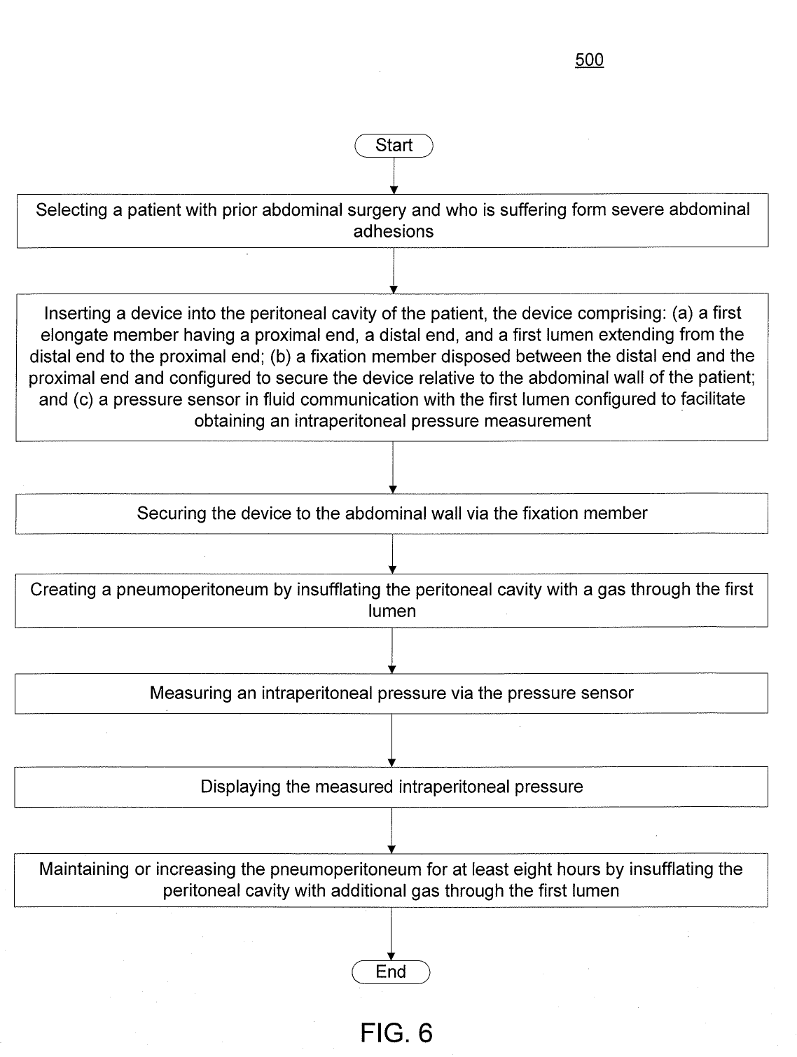

FIG. 6 illustrates an example of a method 500 for creating and maintaining a pneumoperitoneum, in accordance with various aspects of the subject technology. Method 500 comprises selecting a patient with prior abdominal surgery and who is suffering form severe abdominal adhesions and inserting an apparatus 100 into the peritoneal cavity of the patient 180 (e.g., as shown in FIGS. 1 and 2). The apparatus 100 comprises a first elongate member 110a having a proximal end 120a, a distal end 120b, and a first lumen 140a extending from the distal end 130a to the proximal end 130b (e.g., as shown in FIG. 1). The apparatus further comprising a fixation member 150 disposed between the distal end 130a and the proximal end 120a and configured to secure the apparatus 100 relative to the abdominal wall 190 of the patient 180 (e.g., as shown in FIGS. 1 and 2). In some aspects, wherein the first elongate member 110a has a length extending from the distal end 130a to the proximal end 120a, the fixation member 150 is spaced apart from the distal end 130a by at least ⅙ of the length. In some aspects, wherein the first elongate member 110a has a length extending from the distal end 130a to the proximal end 120a, the fixation member 150 is spaced apart from the distal end 130a by at least ¼ of the length. In some aspects, the fixation member 150 is spaced apart from the distal end 130a by at least 10 centimeters. The apparatus 100 further comprising a pressure sensor 50 in fluid communication with the first lumen 140a configured to facilitate obtaining an intraperitoneal pressure measurement. In some aspects, the first elongate member 110a has a diameter of about 5 millimeters, or less. In some aspects, the first elongate member has a diameter of about 2 millimeters or less. In some aspects, the first elongate member 110a comprises a taper that narrows from proximally to distally, thereby facilitating insertion of the first elongate member 110a into the abdomen of the patient 180. In some aspects, a distal portion of the first elongate member 110a comprises a plurality of openings for dissemination of the gas into the peritoneal cavity.

Method 500 also comprises securing the apparatus to the abdominal wall via the fixation member 150 (e.g., as shown in FIGS. 1 and 2). In some aspects, the fixation member 150 comprises a balloon. In some aspects, securing the apparatus comprises inflating the balloon with a fluid.

Method 500 also comprises creating a pneumoperitoneum by insufflating the peritoneal cavity with a gas through the first lumen 140a (e.g., as shown in FIGS. 1 and 2).

Method 500 also comprises measuring an intraperitoneal pressure via the pressure sensor 50 (e.g., as shown in FIG. 1).

Method 500 also comprises displaying the measured intraperitoneal pressure.

Method 500 also comprises maintaining or increasing the pneumoperitoneum for at least eight hours by insufflating the peritoneal cavity with additional gas through the first lumen 140a (e.g., as shown in FIGS. 1 and 2). In some aspects, method 500 comprises at least one of maintaining and increasing the pneumoperitoneum for at least 12 hours by insufflating the peritoneal cavity with additional gas through the first lumen 140a. In some aspects, method 500 comprises at least one of maintaining and increasing the pneumoperitoneum for at least 24 hours by insufflating the peritoneal cavity with additional gas through the first lumen 140a. In some aspects, method 500 comprises at least one of maintaining and increasing the pneumoperitoneum for at least 7 days by insufflating the peritoneal cavity with additional gas through the first lumen 140a. In some aspects, method 500 comprises at least one of maintaining and increasing the pneumoperitoneum for at least 10 days by insufflating the peritoneal cavity with additional gas through the first lumen 140a. In some aspects, method 500 comprises at least one of maintaining and increasing the pneumoperitoneum for at least 20 days by insufflating the peritoneal cavity with additional gas through the first lumen 140a. In some aspects, method 500 comprises at least one of maintaining and increasing the pneumoperitoneum for at least 30 days by insufflating the peritoneal cavity with additional gas through the first lumen 140a.

In some aspects, method 500, comprises sealing the area between the outer surface of the first elongate member 110a and the opening into the peritoneal cavity of the patient 180 with a cover 160 (e.g., as shown in FIGS. 1 and 2). In some aspects, the cover 160 comprises a balloon.

In some aspects, method 500 comprises a valve 170, located at the proximal end 120a, that adjusts a flow of gas through the first elongate member 110a.

In some aspects, method 500 comprises a second elongate member 110b having a proximal portion 120b, a distal portion 130b, and a second lumen 140b extending within the second elongate member 110b (e.g., as shown in FIG. 2). In some aspects, the first and second lumens, 140a and 140b respectively, are in fluid communication with each other. In some aspects, the first and second lumens, 140a and 140b respectively, are in fluid communication with each other proximal to the fixation member 150. In other aspects, the pressure sensor 50 is in fluid communication with the second lumen 140b.

In some aspects, method 500 comprises by a flow meter in fluid communication with the first lumen 140a, measuring a volume of gas flowing through the first lumen 140a. In some aspects, by a counter in fluid communication with the first lumen 140a, counting the number of times the gas flows through the first lumen 140a.

In some aspects, method 500 comprises placing the apparatus 100 into the peritoneal cavity under image guidance by at least one of ultrasound, CT, MRI, and fluoroscopy.

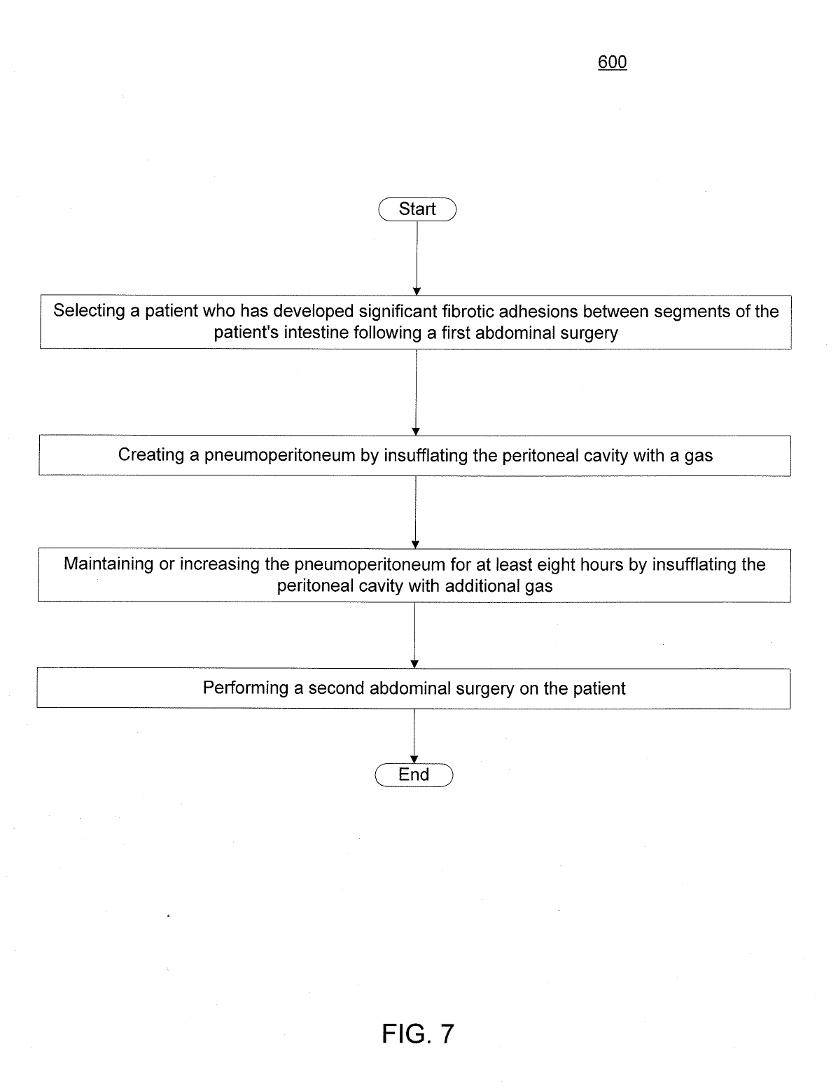

FIG. 7 illustrates an example of a method 600 for treating fibrotic adhesions in a patient, in accordance with various aspects of the subject technology. Method 600 comprises selecting a patient who has developed significant fibrotic adhesions between segments of the patient's intestine following a first abdominal surgery; creating a pneumoperitoneum by insufflating the patient's peritoneal cavity with a gas; maintaining and/or increasing the pneumoperitoneum for at least eight hours by insufflating the peritoneal cavity with additional gas; and performing a second abdominal surgery on the patient.

In some aspects, method 600 comprises at least one of maintaining and increasing the pneumoperitoneum for at least 12 hours by insufflating the peritoneal cavity with additional gas. In some aspects, method 600 comprises at least one of maintaining and increasing the pneumoperitoneum for at least 24 hours by insufflating the peritoneal cavity with additional gas. In some aspects, method 600 comprises at least one of maintaining and increasing the pneumoperitoneum for at least 7 days by insufflating the peritoneal cavity with additional gas. In some aspects, method 600 comprises at least one of maintaining and increasing the pneumoperitoneum for at least 10 days by insufflating the peritoneal cavity with additional gas. For illustration a series of clinical examples are provided.

Case Study No. 1

The patient, a 48-year-old diabetic female, had numerous complications after a gastric bypass procedure originally performed and redone four years later. The patient suffered from a massive midline abdominal hernia associated with chronic pain and fatigue unrelieved by prolonged trials of external support and a variety of medications. The patient had required multiple surgeries for management of intra-abdominal abscesses. The patient also had multiple surgeries for ventral hernia repairs, mesh placement, and mesh explantation. The patient's abdomen was densely adhesed, but nonetheless, the patient required surgical repair of the massive hernia. Subsequent to the repair, the patient had several hospitalizations for wound complications, intra-abdominal abscesses requiring mesh explantation, bowel obstruction and eventually a small bowel fistula. Prolonged total parenteral nutrition (TPN) and a bowel rest program failed to heal the fistula, which presented in the mid-abdomen through the old surgical incision. This incision had broken down and was maintained with vacuum wound management techniques and with wet-to-dry dressing techniques. Finally, with no apparent alternative, the surgeon decided to resect the fistula and, if possible, close the abdomen primarily. Prior to surgery, a central line intravenous catheter was placed as an indwelling intraperitoneal catheter using CT guidance. A progressive pneumoperitoneum was created over the next four days. A total of 15,000 cc of air was installed at the bedside through the catheter using a 50 cc syringe. The patient was then taken to surgery where the fistula and part of the abdominal wall were easily resected. The surgeon did not encounter any adhesions. The pneumoperitoneum technique apparently had lysed the adhesions and made the dissection simple. The surgery, which was originally scheduled for six hours, was completed in less than two hours. The abdominal wall was repaired primarily.

Case Study No. 2

The patient, a 54-year-old female, had an uneventful laparoscopic cholecystectomy. Because of right upper quadrant pain the patient underwent an endoscopic retrograde cholangiopancreatogram (ERCP) six years later, which led to severe ERCP-induced necrotizing pancreatitis. Because of septic complications the patient was taken to surgery for necrosectomy and drainage. In the subsequent six months, twelve abdominal surgeries were performed for debridement of the retroperitoneum, treatment of intra-abdominal hemorrhage and drainage of duodenal contents from a duodenal fistula—all complications of pancreatitis. Several attempts were subsequently made to close the duodenal fistula but the surgeries were limited by the dense adhesions, which prevented either isolating the fistula for diverticularization of the duodenum or creating a Roux-en-Y jejunal loop closure. It was noted that the abdominal contents were encased in such a dense adhesive cocoon that any attempt to dissect the contents would almost surely lead to enterotomies and more fistulae. The surgeon created and maintaining a prolonged pneumoperitoneum, using an intra-abdominal catheter, introduced into the upper abdomen with CT guidance. Approximately 20 liters of air was instilled over seven days at the bedside using a 50 cc syringe. At surgery the adhesions, which were previously noted to be undissectable, were now easily managed. The entire small bowel was dissected, and a Roux-en-Y jejunal loop was created to close the duodenal fistula. A jejunostomy feeding tube and a decompressing biliary T-tube and gastrostomy tube were also placed. Each of these maneuvers had been contemplated during prior surgeries, but were abandoned because of the treacherous nature of the dissection which would have been required. The abdomen was closed primarily.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

A phrase such as an “aspect” does not imply that such aspect is essential to the subject technology or that such aspect applies to all configurations of the subject technology. A disclosure relating to an aspect may apply to all configurations, or one or more configurations. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. A phrase such an embodiment may refer to one or more embodiments and vice versa.

Furthermore, to the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

What is claimed is:

1. A method for creating and maintaining a pneumoperitoneum, the method comprising:
selecting a patient with prior abdominal surgery and who is suffering form severe abdominal adhesions;
inserting a device into the peritoneal cavity of the patient, the device comprising: (a) a first elongate member having a proximal end, a distal end, and a first lumen extending from the distal end to the proximal end; (b) a fixation member disposed between the distal end and the proximal end and configured to secure the device relative to the abdominal wall of the patient; and (c) a pressure sensor in fluid communication with the first lumen configured to facilitate obtaining an intraperitoneal pressure measurement;
securing the device to the abdominal wall via the fixation member;
creating a pneumoperitoneum by insufflating the peritoneal cavity with a gas through the first lumen;
measuring an intraperitoneal pressure via the pressure sensor;
displaying the measured intraperitoneal pressure; and
maintaining or increasing the pneumoperitoneum for at least eight hours by insufflating the peritoneal cavity with additional gas through the first lumen.

2. The method of claim 1, further comprising at least one of maintaining and increasing the pneumoperitoneum for at least 12 hours by insufflating the peritoneal cavity with additional gas through the first lumen.

3. The method of claim 1, further comprising at least one of maintaining and increasing the pneumoperitoneum for at least 24 hours by insufflating the peritoneal cavity with additional gas through the first lumen.

4. The method of claim 1, further comprising at least one of maintaining and increasing the pneumoperitoneum for at least 7 days by insufflating the peritoneal cavity with additional gas through the first lumen.

5. The method of claim 1, further comprising at least one of maintaining and increasing the pneumoperitoneum for at least 10 days by insufflating the peritoneal cavity with additional gas through the first lumen.

6. The method of claim 1, wherein the fixation member comprises a balloon.

7. The method of claim 6, wherein the securing the device comprises inflating the balloon with a fluid.

8. The method of claim 1, wherein the first elongate member has a length extending from the distal end to the proximal end, and the fixation member is spaced apart from the distal end by at least ⅙ of the length.

9. The method of claim 1, wherein the first elongate member has a length extending from the distal end to the proximal end, and the fixation member is spaced apart from the distal end by at least ¼ of the length.

10. The method of claim 1, wherein the fixation member is spaced apart from the distal end by at least 10 centimeters.

11. The method of claim 1, further comprising sealing the area between the outer surface of the first elongate member and the opening into the peritoneal cavity of the patient with a cover.

12. The method of claim 11, wherein the cover comprises a balloon.

13. The method of claim 1, further comprising a valve, located at the proximal end, that adjusts a flow of gas through the first elongate member.

14. The method of claim 1, wherein the first elongate member comprises a taper that narrows from proximally to distally, thereby facilitating insertion of the first elongate member into the abdomen of the patient.

15. The method of claim 1, wherein the first elongate member has a diameter of about 5 millimeters or less.

16. The method of claim 1, wherein the first elongate member has a diameter of about 2 millimeters or less.

17. The method of claim 1, wherein a distal portion of the first elongate member comprises a plurality of openings for dissemination of the gas into the peritoneal cavity.

18. The method of claim 1, wherein the device further comprises a second elongate member having a proximal portion, a distal portion, and a second lumen extending within the second elongate member.

19. The method of claim 18, wherein the first and second lumens are in fluid communication with each other.

20. The method of claim 18, wherein the first and second lumens are in fluid communication with each other proximal to the fixation member.

21. The method of claim 18, wherein the pressure sensor is in fluid communication with the second lumen.

22. The method of claim 1, further comprising by a flow meter in fluid communication with the first lumen, measuring a volume of gas flowing through the first lumen.

23. The method of claim 1, further comprising by a counter in fluid communication with the first lumen, counting the number of times the gas flows through the first lumen.

24. The method of claim 1, further comprising placing the device into the peritoneal cavity under image guidance by at least one of ultrasound, CT, MRI, and fluoroscopy.

25. An apparatus for creating and maintaining a pneumoperitoneum, the apparatus comprising:
a first elongate member having a proximal end, a distal end, and a first lumen extending from the distal end to the proximal end;
a fixation member disposed between the distal end and the proximal end and configured to secure the apparatus relative to the abdominal wall of a patient;
a pressure sensor in fluid communication with the first lumen configured to facilitate obtaining an intraperitoneal pressure measurement;
a valve, located at the proximal end and configured to adjust a flow of gas through the first elongate member; and
a flow meter in fluid communication with the first lumen and configured to measure a volume of gas flowing through the first lumen.

26. The apparatus of claim 25, wherein the fixation member comprises a balloon.

27. The apparatus of claim 26, wherein the balloon is inflatable with a fluid.

28. The apparatus of claim 25, wherein the first elongate member has a length extending from the distal end to the proximal end, and the fixation member is spaced apart from the distal end by at least ⅙ of the length.

29. The apparatus of claim 25, wherein the first elongate member has a length extending from the distal end to the proximal end, and the fixation member is spaced apart from the distal end by at least ¼ of the length.

30. The apparatus of claim 25, wherein the fixation member is spaced apart from the distal end by at least 10 centimeters.

31. The apparatus of claim 25, further comprising a cover configured to seal the area between the outer surface of the first elongate member and the opening into the peritoneal cavity of the patient.

32. The apparatus of claim 31, wherein the cover comprises a balloon.

33. The apparatus of claim 25, wherein the first elongate member comprises a taper that narrows from proximally to distally, thereby facilitating insertion of the first elongate member into the abdomen of the patient.

34. The apparatus of claim 25, wherein the first elongate member has a diameter of about 5 millimeters or less.

35. The apparatus of claim 25, wherein the first elongate member has a diameter of about 2 millimeters or less.

36. The apparatus of claim 25, wherein a distal portion of the first elongate member comprises a plurality of openings for dissemination of the gas into the peritoneal cavity.

37. The apparatus of claim 25, further comprising a second elongate member having a proximal portion, a distal portion, and a second lumen extending within the second elongate member.

38. The apparatus of claim 37, wherein the first and second lumens are in fluid communication with each other.

39. The apparatus of claim 37, wherein the first and second lumens are in fluid communication with each other proximal to the fixation member

40. The apparatus of claim 37, wherein the pressure sensor is in fluid communication with the second lumen.

41. The apparatus of claim 25, wherein the first elongate member is at least partially radiopaque.

42. An apparatus for creating and maintaining a pneumoperitoneum, the apparatus comprising:
a first elongate member having a proximal end, a distal end, and a first lumen extending from the distal end to the proximal end;
means for securing the apparatus relative to the abdominal wall of a patient;
a valve located at the proximal end and configured to adjust a flow of gas through the first elongate member;
means for measuring an intraperitoneal pressure; and
means for measuring a volume of gas flowing through the first lumen.

43. The apparatus of claim 42, wherein the means for securing comprises a balloon disposed between the distal end and the proximal end.

44. The apparatus of claim 43, wherein the first elongate member has a length extending from the distal end to the proximal end, and the balloon is spaced apart from the distal end by at least ⅙ of the length.

45. The apparatus of claim 42, wherein the first elongate member has a length extending from the distal end to the proximal end, and the balloon is spaced apart from the distal end by at least ¼ of the length.

46. The apparatus of claim 42, wherein the balloon is spaced apart from the distal end by at least 10 centimeters.

47. The apparatus of claim 42, wherein the balloon is inflatable with a fluid.

48. The apparatus of claim 42, further comprising a cover configured to seal the area between the outer surface of the first elongate member and the opening into the peritoneal cavity of the patient.

49. The apparatus of claim 48, wherein the cover comprises a balloon.

50. The apparatus of claim 42, wherein the first elongate member comprises a taper that narrows from proximally to distally, thereby facilitating insertion of the first elongate member into the abdomen of the patient.

51. The apparatus of claim 42, wherein the first elongate member has a diameter of about 5 millimeters or less.

52. The apparatus of claim 42, wherein the first elongate member has a diameter of about 2 millimeters or less.

53. The apparatus of claim 42, wherein a distal portion of the first elongate member comprises a plurality of openings for dissemination of the gas into the peritoneal cavity.

54. The apparatus of claim 42, wherein the means for measuring an intraperitoneal pressure comprises a pressure sensor in fluid communication with the first lumen.

55. The apparatus of claim 42, wherein the means for measuring an intraperitoneal pressure comprises a second elongate member having a proximal portion, a distal portion, and a second lumen extending within the second elongate member, the distal portion configured to facilitate obtaining an intraperitoneal pressure measurement displayed at the proximal portion.

56. The apparatus of claim 55, wherein the first lumen and second lumen are in fluid communication with each other.

57. The apparatus of claim 55, wherein the first lumen and second lumen are in fluid communication with each other proximal to the fixation member.

58. The apparatus of claim 42, wherein the means for measuring a volume of gas flowing through the first lumen comprises a flow meter in fluid communication with the first lumen and configured to measure a volume of gas flowing through the first lumen.

59. The apparatus of claim 42, wherein the means for measuring a volume of gas flowing through the first lumen comprises a counter in fluid communication with the first lumen and configured to count the number of times the gas flows through the first lumen.

60. The apparatus of claim 42, wherein the first elongate member is at least partially radiopaque.

61. A method for treating fibrotic adhesions in a patient, the method comprising:
selecting a patient who has developed significant fibrotic adhesions between segments of the patient's intestine following a first abdominal surgery;
creating a pneumoperitoneum by insufflating the patient's peritoneal cavity with a gas;
prior to performance of a second abdominal surgery on the patient, maintaining and/or increasing the pneumoperitoneum for at least eight hours by insufflating the peritoneal cavity with additional gas.

62. The method of claim 61, further comprising at least one of maintaining and increasing the pneumoperitoneum for at least 12 hours by insufflating the peritoneal cavity with additional gas.

63. The method of claim 61, further comprising at least one of maintaining and increasing the pneumoperitoneum for at least 24 hours by insufflating the peritoneal cavity with additional gas.

64. The method of claim 61, further comprising at least one of maintaining and increasing the pneumoperitoneum for at least 7 days by insufflating the peritoneal cavity with additional gas.

65. The method of claim 61, further comprising at least one of maintaining and increasing the pneumoperitoneum for at least 10 days by insufflating the peritoneal cavity with additional gas.
